(19) United States
(12) Patent Application Publication
(10) Pub. No.: US 2025/0359906 A1
(43) Pub. Date: Nov. 27, 2025

(54) SYSTEMS FOR SURGICAL TREATMENT OF THE ELBOW

(71) Applicant: Shahryar AHMADI, Huntington Beach, CA (US)

(72) Inventor: Shahryar AHMADI, Huntington Beach, CA (US)

(21) Appl. No.: 19/211,865

(22) Filed: May 19, 2025

Related U.S. Application Data
(60) Provisional application No. 63/650,033, filed on May 21, 2024.

(51) Int. Cl. A61B 17/86; A61B 17/56

(52) U.S. Cl. CPC A61B 17/86; A61B 2017/564; A61B 2017/567

(57) ABSTRACT

Orthopedic implants, surgical tools and surgical methods. More particularly, a system for surgical treatment of the elbow region for various fractures. The implants include an assortment of humerus plates, an olecranon plate system, and an elbow stabilization system. In order to assist in surgery, the system includes guides for the humerus, a coronoid fracture reduction clamp and an olecranon osteotomy clamp. Methods of using the various devices are also disclosed.

### FIELD OF THE INVENTION

The present invention relates to orthopedic implants, surgical tools and surgical methods. More particularly, the invention relates to a system for surgical treatment of the elbow region for various fractures. The implants include an assortment of humerus plates, an olecranon plate system, and an elbow stabilization system. In order to assist in surgery, the system includes guides for the humerus, a coronoid fracture reduction clamp and an olecranon osteotomy clamp. Methods of using the various devices are also disclosed.

### BACKGROUND OF THE INVENTION

The bones of the elbow region, namely the radius and ulna of the forearm and humerus of the upper arm, are vulnerable to fractures which can be caused by falling directly on the elbow, receiving a direct blow to the elbow from a hard object, for example a dashboard or car door during a vehicle collision, or falling on an outstretched arm with the elbow held tightly to brace against the fall. The humerus, which is the upper arm bone, radius, which is the outer forearm bone located on the thumb side of the forearm and the ulna, which is the inner forearm bone located on the pinky side of the arm, together form the elbow joint.

Three primary types of elbow fractures occur including olecranon fractures, radial head fractures and distal humerus fractures.

The olecranon is the area of ulnar bone at the tip of the elbow. The olecranon is vulnerable to fracture because it is not covered by any substantial muscle or other tissue.

The head of the radius connects with the humerus and can fracture when a person tries to break a fall with their arm. As a result of a fall, the radial head pushes into the humerus and causes a fracture.

The distal humerus is the rounded bottom part of the bone connecting the shoulder to the elbow. Fractures of the distal humerus occur as a result of a direct blow to the elbow, such as by extending a hand out to break a fall, or falling on a bent elbow. Fractures of the distal humerus are less common than the primary elbow fracture types.

Increased risks of elbow region fractures occur in individuals who have osteoporosis, older adults, women after menopause, individuals who have a condition resulting in bone loss, weak bones or osteoporosis, for example.

Various complications can arise from elbow fractures including loss of range of motion, stiffness in the elbow joint, development of arthritis, and misalignment of the elbow joint.

In order to return or maintain function of the elbow, bone fractures need to be repaired quickly and correctly. Fractured bones are often treated using fixation devices, which align bone fragments during healing and provide reinforcement to the affected bone. Such fixation devices include external fixation devices such as casts and internal fixation devices such as plates or plating systems as well as stabilization systems, such as available from Skeletal Dynamics as an IJS device.

Bone plate systems include plates that are typically metal and manipulated or bent by a surgeon in order to conform to a region of bone spanning a fracture and subsequently fastened to the bone on opposite sides of the fracture utilizing a suitable fastener, such as one or more of screws and wires, which hold the fractured bone together during and possibly after healing. Bone plates have been provided in various sizes, shapes, lengths and widths in order to accommodate various sizes and shapes of bones.

Bone plates are often the treatment of choice by a surgeon and allow relatively rapid return to motion. Nevertheless, bone plates known in the art have some disadvantages. Certain areas of bones, for example, the distal end of the humerus may require the use of more than one plate, especially if the fracture of bone is relatively complex. Furthermore, setting a fracture using more than one plate may be complicated especially if the plates need to be contoured to a complex shape prior to use.

Some stabilization systems meant to deal with elbow instability also exhibit problems in some instances when they include pins that can wobble and damage the bone. In addition, some systems cannot be used in conjunction with bone plates. Still other systems suffer from drawbacks as implants are connected where one or more collateral ligaments are attached. This causes nonisometric repair of collateral ligament and potential damage to collateral ligament, residual elbow instability and/or elbow stiffness.

In view of the above, the art still needs improved plating system for surgical treatment of the elbow, as well as elbow stabilization systems. In addition, surgical guides and clamps for aiding surgical procedures are also needed.

### SUMMARY OF THE INVENTION

The problems of the prior art and others are solved with the system and methods of surgery using the system of the present invention for treating elbow fractures. The system includes an elbow stabilization system including a humerus plate and an ulna plate system interconnected by an adjustable link hingedly connected to the humerus plate. The system further includes a distal humerus lateral plate system, a distal humerus medial plate system, and an olecranon plate system for fixing various fractures of the respective bones. The surgical implements of the system include a surgical drill guide for the humerus, a surgical clamp for the ulna that can be used to reduce a coronoid fracture, as well as an olecranon osteotomy clamp. Methods for using the devices and tools are disclosed as well.

In one embodiment, the components described herein are provided as an elbow surgical repair kit, allowing the surgeon to select one or more components from the set necessary for fixing a particular fracture or fractures. Advantageously, the components of the kit can be used together and work in synergy.

In a further embodiment, an elbow stabilization system is disclosed, comprising a humerus plate adapted to be connected to an external surface at a distal end of the humerus, wherein the plate has an upper proximal segment that includes a plurality of apertures adapted for fasteners that connect the humerus plate to the humerus, an adjustable link hingedly connected to the humerus plate and having a hinge adapted to be a located adjacent where the lateral collateral ligament attaches to the humerus, wherein the adjustable link is independently adjustable in length and at least two different angles and also able to be subsequently locked in length and angle after a desired length and angle have been established; and an ulna plate system comprising an inner plate and an outer plate each including a pair of apertures disposed at opposite ends of the plate and adapted for fasteners to fix the plates to the ulna, wherein an ulna end of the adjustable link is fixedly connectable to the ulna inner and outer plates.

In yet another embodiment, a distal humerus lateral plate system is disclosed, comprising the plate having a longitudinal length and a width narrower than the length, the plate having an upper proximal first end and a lower distal second end, wherein a lower portion of the plate curves outwardly to adapt to a contour of the humerus above a lateral epicondyle of the humerus, wherein the second end of the plate includes an inwardly curved section adapted to the contour of the lateral epicondyle, wherein the plate has a plurality of vertically spaced apertures for fasteners along the longitudinal length of the plate.

In still another embodiment, a distal humerus medial plate system is disclosed, comprising the plate having a longitudinal length and a width narrower than the length, the plate having an upper proximal first end and a lower distal second end, wherein a lower portion of the plate curves outwardly to adapt to a contour of the humerus above the medial epicondyle of the humerus, and wherein the plate has a plurality of vertically spaced apertures for fasteners along the longitudinal length of the plate.

In a further embodiment, a surgical drill guide for the humerus is disclosed, comprising a first frame having a first end having a hollow elongated drill guide having a bore extending therethrough, and a second frame having a first end having a point that aligns with the bore extending through the drill guide, wherein the drill guide includes a locking mechanism that fixes the distance between the drill guide and the point, wherein the locking mechanism is releasable after use in order to reposition the first frame with respect to the second frame.

In yet another embodiment, an olecranon osteotomy clamp and guide is disclosed, comprising a first arm hinged to a second arm, wherein each arm has a handle, wherein each arm has a jaw at an end opposite from the handle, which provide a guide for both straight and chevron osteotomy; and a locking mechanism that secures the arms in a fixed position.

In still another embodiment, a olecranon plate system is disclosed, comprising an olecranon plate having a head segment, a stem segment connected to and extending from the head segment, an inner surface adapted to engage bone of an ulna, and an outer surface opposite the inner surface, wherein the head segment includes an outer rim having a head inner surface, a head outer surface and a central opening within the head inner surface, wherein the outer rim includes an olecranon cradle adapted to be situated at the head of the olecranon, wherein the olecranon cradle includes a plurality of apertures each adapted for receiving a fastener, wherein a plurality of apertures are present in the outer rim spaced about the central opening, wherein a web is present in the central opening, with the web having a plurality of wires that connect the web to the inner surface of the rim, wherein the web includes a plurality of apertures adapted for receiving the fasteners, wherein at least one portion of the web can be removed from the plate by cutting the web wires, and wherein the stem segment includes a plurality of apertures adapted for receiving fasteners.

In a further embodiment, a surgical clamp for the ulna suitable for use in reducing a coronoid fracture is disclosed, comprising a first arm hinged to a second arm, with each arm having a handle at a first end, wherein the first arm has a head at a second end opposite the first end, wherein the head includes a coronoid cup adapted to accommodate a portion of the coronoid, wherein the cup has a rim and a base recessed within the rim, wherein the second arm has a hollow elongated drill guide tube having a bore extending therethrough at a second end thereof, and wherein a line extending through a longitudinal axis of the drill guide tube also passes to the base of the head of the first arm with the first arm located at a first position with respect to the second arm.

### BRIEF DESCRIPTION OF THE DRAWINGS

The invention will be better understood, and other features and advantages will become apparent by reading the detailed description of the invention, taken together with the drawings, wherein:

FIG. 1 is a side, lateral view of a right elbow region repaired with an elbow stabilization system of one embodiment of the invention including a humerus plate, an adjustable link hingedly connected to the humerus plate, and an ulna plate system;

FIG. 2 is a front view showing the humerus plate connected to an external surface at a distal end of the humerus with two fasteners, as well as a K-wire and sleeve that aid in placement of the humerus plate;

FIG. 3 is front view of the humerus plate and a portion of the adjustable link particularly illustrating the hinged connection;

FIG. 4 illustrates the ulna plate of the elbow stabilization system in an unassembled configuration, as well as after assembly wherein the two plates are connected with fasteners;

FIG. 5 illustrates the humerus plate of the elbow stabilization system with the outer segment of the rotating part removed to show the rotating assembly;

FIG. 6 illustrates a front view of a humerus having one embodiment of a distal humerus lateral plate system and distal humerus medial plate system attached thereto;

FIG. 7 illustrates side views of the distal humerus lateral plate A) without an extension, B) with a posterior extension, the distal humerus medial plate C) with a medical epicondyle wraparound segment and D) without a wraparound segment;

FIG. 8 illustrates side views of A) the distal humerus lateral plate without an extension, B) the distal humerus lateral plate system with posterior extension and C) the distal humerus medial plate system without extension;

FIG. 9 illustrates a front view of a surgical guide for the humerus being used therewith;

FIG. 10 illustrates A) one embodiment of an olecranon osteotomy clamp in use on an ulna to cut a portion of the olecranon and B) illustrating the olecranon after osteotomy;

FIG. 11 is a lateral side view of one embodiment of an olecranon plate system of the present invention;

FIG. 12 is a further embodiment of the olecranon plate system including a web present in a central opening;

FIG. 13 is a view of the olecranon plate system with the web removed;

FIG. 14 illustrates a further embodiment of an olecranon plate system A) in a bottom view, B) in a side view and C) in a further side view;

FIG. 15 illustrates A) one embodiment of a surgical clamp for the ulna suitable for reducing a coronoid fracture, B) an enlarged view showing a second arm of the drill guide having a hollow elongated drill guide tube having a bore extending therethrough in conjunction with a K-wire and C), the K-wire fixed in place with the surgical drill guide removed; and

FIG. 16 illustrates A) the coronoid clamp in use on an ulna to reduce a coronoid fracture and B) a portion of an ulna having a coronoid fracture.

### DETAILED DESCRIPTION OF THE DRAWING

This description of preferred embodiments is to be read in connection with the accompanying drawings, which are part of the entire written description of this invention. In the description, corresponding reference numbers are used throughout to identify the same or functionally similar elements. Relative terms such as “horizontal,” “vertical,” “up,” “upper”, “down,” “lower”, “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and are not intended to require a particular orientation unless specifically stated as such. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship.

The invention relates to a system for surgical treatment of fractures around the elbow region including stabilization devices including an elbow stabilization system, a distal humerus lateral plate system, a distal humerus medial plate system and an olecranon plate system, each of which aid a surgeon in reducing, fixating or both, bone fractures. In addition, devices for assisting with surgical treatment are provided, namely surgical guides for the humerus and an olecranon osteotomy clamp.

Still further, an elbow surgical repair kit is disclosed comprising bone plates, stabilization systems and surgical tools for surgical treatment.

Methods of surgical treatment and use of the components are described herein as well.

### Elbow Stabilization System

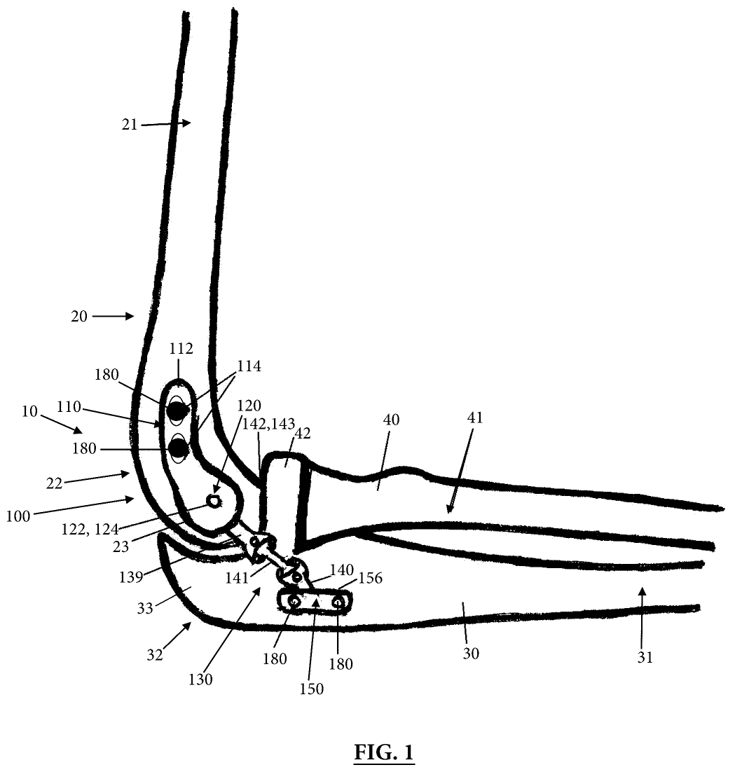

Referring now to FIGS. 1 through 5, various components of an elbow stabilization system 104 and elbow 10 are illustrated. FIG. 1 shows the elbow stabilization system 100 in place on an elbow 10, with a right elbow shown in a side, lateral view, a bent elbow with forearm in neutral rotation configuration with the hand, not shown. The view includes the distal humerus 20, the proximal portion of ulna 30 and the proximal portion of radius 40. The humerus 20 includes a shaft region 21 and an end region 22. The ulna 30 includes a shaft region 31, end region 32 including olecranon 33. Radius 40 also includes a shaft region 41 and end region 42.

Elbow stabilization system 100 includes a humerus plate 110 adapted to be connected to humerus 20 with a plurality of fasteners 180 such as but not limited to surgical screws of appropriate sizes, an adjustable link 130 hingedly connected to humerus plate 110, and ulna plate 150 adapted to be fixed to the ulna 30 with two or more fasteners 180.

Humerus plate 110 can be connected directly to humerus 20 or indirectly to the humerus through the distal humerus lateral plate 300 described further herein. As illustrated, the elbow stabilization system 100 is an internal device for keeping the elbow stable through the range of motion that does not compromise the collateral ligament. System 100 is designed so that it can also be applied in fracture cases right on the plate. In addition, when utilized correctly the system does not cause any bone destruction. The device is quite sturdy and stable, especially compared to other prior art systems. The elbow stabilization system 100 is relatively facile to install and remove. Advantageously, it can be adjusted to substantially any anatomy.

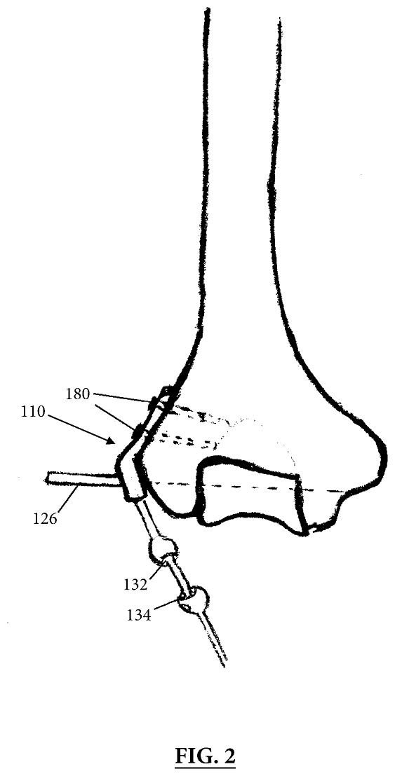

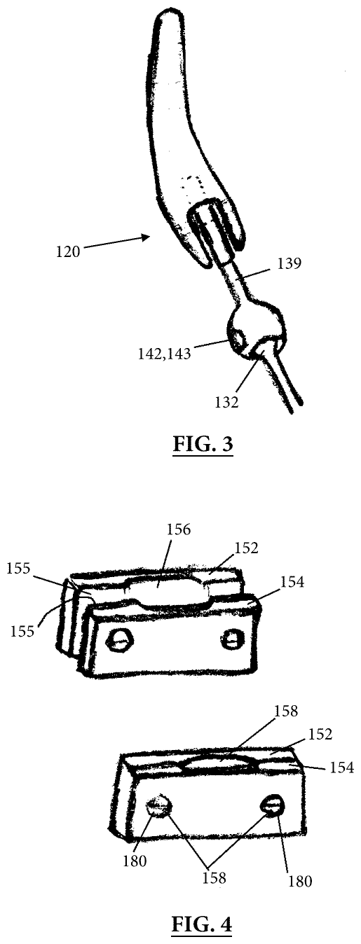

As shown between FIGS. 1 and 2, the humerus plate 110 includes an upper, proximal segment 112 having a pair of apertures 114, preferably oblong apertures, that allow precise alignment and fitment when utilized with a distal humerus lateral plate 300. The fasteners 180 can secure the humerus plate 110 in any desired position, even when the distal humerus lateral plate 300 is not utilized or necessary. Lower distal segment 116 is angled forward with respect to the upper proximal segment 112. Lower distal segment 116 as shown in FIG. 3 includes a housing 120 that hingedly connects adjustable link 130 thereto. Humerus plate 110 is connected to the humerus 20 at a location such that hinge point 122 between plate 110 and adjustable link 130 is adjacent the location where the lateral collateral ligament attaches to the humerus. This positioning maintains isometric movement of the elbow.

Adjustable link 130 is independently adjustable in length and angle and also able to be subsequently locked in both length and angle after a desired orientation has been established based upon the anatomy of the patient. The adjustable link comprises two separate ball and socket assemblies comprising a first ball 132 and a second ball 134, see FIGS. 1, 2, 3 and 5, as well as a first socket 136 and a second socket 138. A first pin 139 is connected to first socket 136 and a second pin 140 is connected to second socket 138.

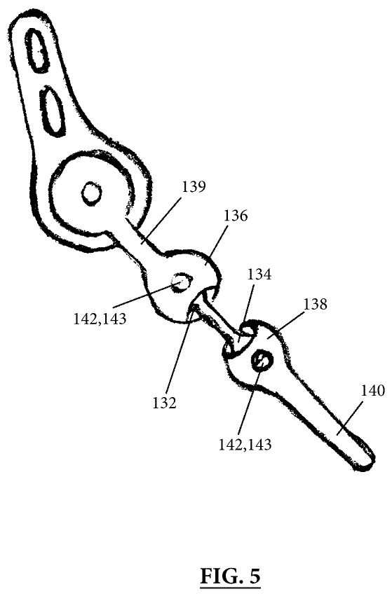

First pin 139 is hingedly connected to humerus plate 110. FIG. 3 shows stabilizing device from front. The plate includes housing 120 that is hinged to a pin 139. FIG. 5 shows the device from the side with part of plate removed to show the hinge.

Second pin 140 is fixable in slot 146 of the ulna plate system 150 as described below.

In a preferred embodiment, connecting link 141 is provided having the first ball 132 at a first end and the second ball 134 at a second end thereof. Thus, in one embodiment, the connecting link is essentially or substantially shaped like a dog bone with balls at each end. Each socket 136, 138 has a socket aperture 142 that is preferably a threaded bore which accepts a fastener 143, preferably a set screw, that is used to secure a respective ball in a respective socket thereby preventing rotation between the pair.

The ulna plate system 150 comprises an inner plate 152 and an outer plate 154. Each plate 152, 154 includes two or more apertures 158 that accept fasteners 180 for securing the inner plate 152 and outer plate 154 to ulna 30. One or more of the plates include a slot 156 formed in a sidewall 155 thereof which accept a portion of second pin 140 therein. Once the pin is situated at a desired location within slot 156, the fasteners 180 are tightened thereby fixing and anchoring the ulna end of second pin 140 within slot 156.

In an alternative embodiment, second pin 140 includes an aperture at a distal end thereof, opposite second socket 138 that can be utilized to attach the pin 140 directly to the ulna.

In still a further embodiment, adjustable link 130 includes a pair of sockets at each end and balls are present on each of the pins in place of first socket 136 and second socket 138.

### Surgical Drill Guide

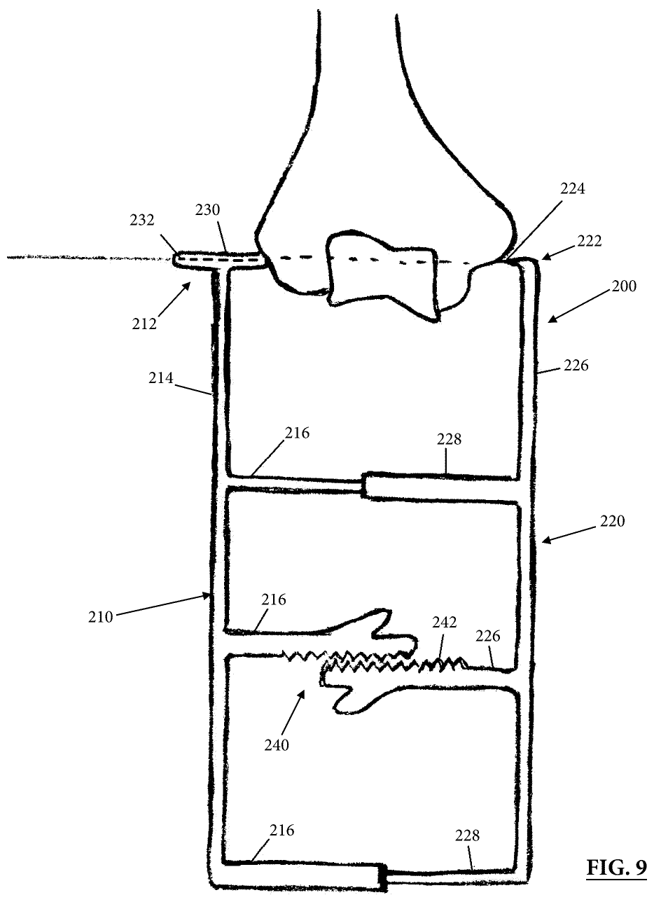

The humerus plate is preferably installed utilizing the surgical drill guide 200 illustrated in FIG. 9. Surgical drill guide 200 includes a lateral or first frame 210 and a medial or second frame 220. The first frame has a first end 212 having a hollow elongated drill guide 230 having a bore 232 extending therethrough. In a preferred embodiment, bore 232 is perpendicular to a longitudinal length of first frame 210. Second frame 220 has a first end 222 having a projection or point 224 that aligns with the longitudinal length or axis of bore 232 of drill guide 230. Stated in another manner, a straight line extending through bore 232 of drill guide 230 also runs through point 224 of second frame 220. This aids in the surgeon aligning the humerus plate with the anatomy of the patient.

The drill guide 200 includes a locking mechanism 240 that fixes the distance between the drill guide 230 and point 224. The locking mechanism is releasable after use in order to separate the first frame and the second frame and remove the surgical drill guide 200 from the bone to which it was previously locked. First frame 210 includes a body 214 extending substantially perpendicular to the longitudinal length of the drill guide 230. An arm 216 and preferably a plurality of arms 216 extend outwardly from the body, with each arm 216 having a longitudinal length. The second frame 220 also includes a body 226 extending substantially perpendicular to the longitudinal length of the drill guide 230, with at least one arm 228 extending outwardly preferably substantially perpendicular from the body 226. Arm 228 of second frame 220 also has a longitudinal length. The one or more first frame arms 216 and second frame arms 228 are telescopically connected in a preferred embodiment. As illustrated in FIG. 9, at least one arm 228 or 216 is formed as a tube and the opposite arm 216 or 228 respectively is formed as a pin which is insertable in the other arm and moveable therewithin in order to adjust lateral distance between body 214 and body 226.

Locking mechanism 240 preferably includes teeth 242 or some other functionally equivalent structure on each first frame locking mechanism arm and second frame locking mechanism arm that are releasably interlockable.

In some embodiments, drill guide 230 is hingedly connected to first frame 210 through a suitable hinge.

### Humerus Plate Attachment

With reference to FIGS. 2 and 9, one method for attaching the humerus plate of the elbow stabilization system 100 to the humerus 20 is described. First, surgical drill guide 200 is operatively connected to the humerus 20. Drill guide 230 is aligned with and engages the lateral collateral ligament origin and the point 224 of second frame 220 is engaged with the origin of the medial collateral ligament (axis of rotation of elbow joint). A hole is drilled in the humerus 20 through the drill guide. A K-wire or other surgical wire is inserted into the humerus hole through drill guide 230.

As illustrated in FIG. 2, the humerus plate central aperture 124 has a sleeve removably attached thereto, such as through a threaded connection. With the K-wire serving as a guide, the humerus plate 100 aligned with the humerus, with the sleeve 126 being positioned over the K-wire. The humerus plate central aperture 124 which has a sleeve 126 removably attached thereto. Humerus plate 110 is then fixed to humerus 20 utilizing fasteners 180 as illustrated in the drawings. Thereafter, the sleeve 126 and K-wire are removed from plate 110.

The surgical drill guide 200 can also be used to aid in placing fasteners through the plate 110. By using the sharp point 224 of the drill guide 200 on the other side of the bone and the drill guide 230 on the aperture of the plate 110, one can predict the direction of the drill and screw to prevent any unsatisfactory placement of the screw.

### Angle and Length Adjustment

After the humerus plate 110 has been connected to the humerus 20, the ulnar plate is applied on the lateral side of proximal ulna. The ulna end of second pin 140 is disposed within slot 156 of the ulna plate system 150. The elbow is reduced and kept reduced. Then, the inner plate 152 and outer plate 154 are fastened with fasteners 180 to ulna 30, locking the adjustable link 130 in place. Also each of the balls 132, 134 and sockets 136, 138 are adjusted and fixed.

### Distal Humerus Lateral Plate System

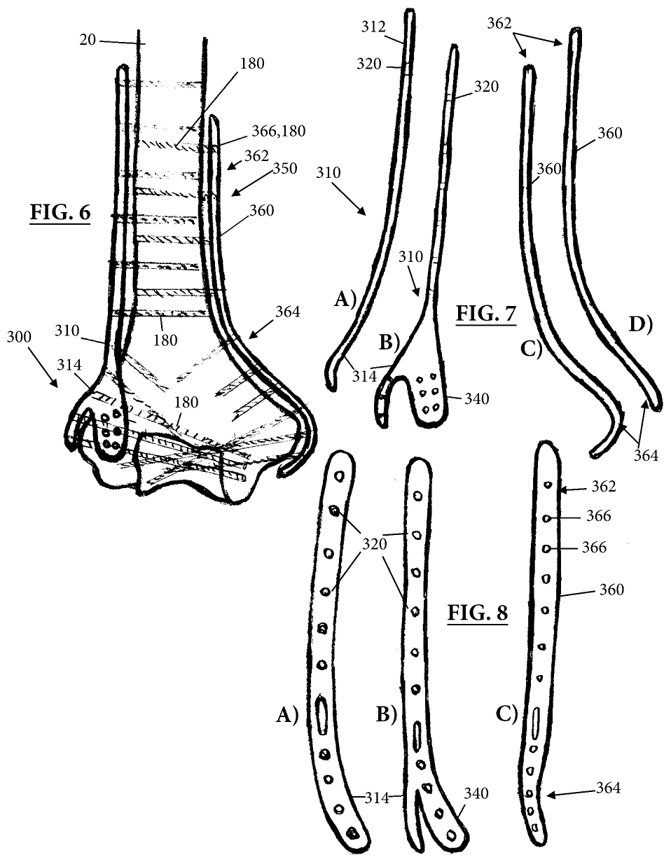

FIGS. 6-8 show various embodiments of a distal humerus lateral plate system 300 including distal humerus lateral plate 310. Lateral plate 310 is engineered so that it sits adjacent to and is affixed on the lateral side of the humerus 20. In one embodiment, the lateral plate 310 has a posterior extension 340 which extends distally over the posterior part of the humerus. At times, when a surgeon fixes a humerus fracture, it will look very good on an anterior to posterior view of the x-ray, but in a lateral view the distal part of the humerus may be in an extended position. The posterior extension 340 extends over the posterior part of the humerus to aid in reduction and bring the distal humerus back to an anatomic angle.

The lateral plate 310 has a longitudinal length extending generally in the direction the length of the humerus, and a width narrower than the length. The lateral plate 310 has an upper proximal first end 312 and a lower distal second end 314. A lower portion of the lateral plate 310 curves outwardly to adapt to a contour of the humerus above the lateral epicondyle of the humerus. Below the outward curve, the second end 314 of the lateral plate 310 curves inwardly and is adapted to the contour of the lateral epicondyle. The lateral plate 310 has a plurality of apertures 320 which are vertically spaced along the longitudinal length of the lateral plate 310 which accept fasteners 180 for fixing the lateral plate 310 to the humerus at a desired location.

When the second end 314 of the lateral plate 310 includes the posterior extension 340, the latter is adapted to a contour of a posterior part of the humerus 20. In a preferred embodiment, the posterior extension 340 also includes a plurality of spaced apertures 320 for fasteners 330. In some embodiments, the posterior extension includes three sets of two laterally adjacent apertures vertically spaced along the longitudinal length thereof. In some embodiments, one or more of the apertures are oblong which aids in adjusting placement of the lateral plate on the humerus.

The versatility of the plate provides an option for fasteners such as 2.4 or 2.7 mm screws that can be inserted through the plate distally and either 2.7 mm or 3.5 mm screws proximally.

FIGS. 7A and 8A show the lateral plate 310 without any posterior extension. FIGS. 7B and 8B show the lateral plate 310 with posterior extension 340. Posterior extension is also very helpful for fixation capitellum.

In some embodiments, the plates have a thickness that ranges between 2.7 mm and 3.5 mm.

### Distal Humerus Medial Plate System

FIGS. 6-8 also show various embodiments of a distal humerus medial plate system 350 including a distal humerus medial plate 360 and fasteners 180 which comprise the system. Medial plate 360 is designed so that it sits adjacent to and is affixed on the medial side of the humerus 20. The medial plate 360 has a longitudinal length extending generally in the direction of the length of the humerus and a width narrower than the length. The medial plate 360 has an upper proximal first end 362 and a lower distal second end 364. A lower portion of the medial plate 360 first curves outwardly to adapt to the contour of the humerus above the medial epicondyle. Optionally, below the outward curve, the second end 364 curves back inwardly and is adapted to more or less wrap around at least a portion of the medial epicondyle.

The medial plate 360 has a plurality of apertures 366 which are vertically spaced along the longitudinal length of the medial plate 360 which accepts fasteners 180 for fixing the medial plate 360 to the humerus 20 at a desired location. In some embodiments, one or more of the apertures are oblong which aids in adjusting placement of the medial plate on the humerus.

### Olecranon Osteotomy Clamp and Guide

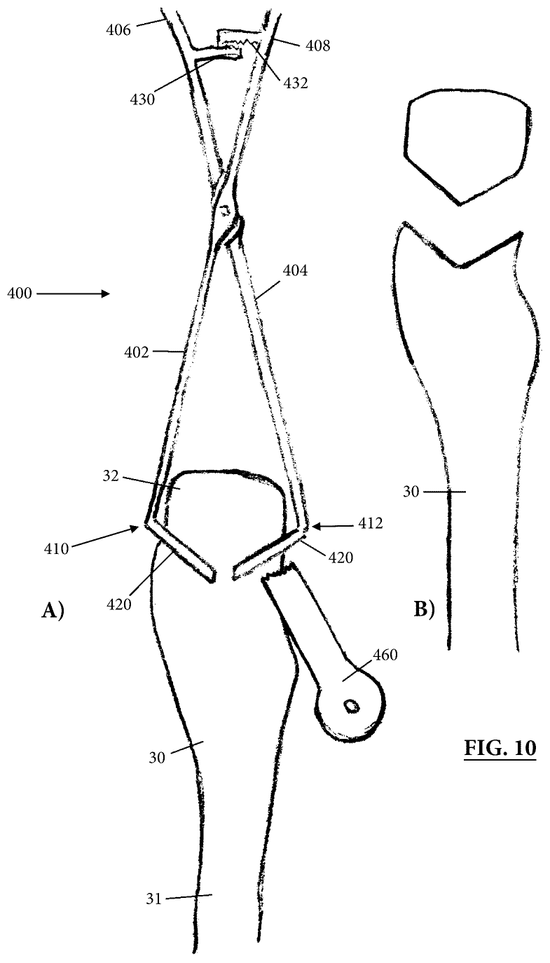

It is occasionally necessary for complex and comminuted distal humerus fractures to perform an olecranon osteotomy whereby at least a portion of the olecranon is mobilized from the proximal head of the ulna 30. The procedure can be a challenge for the surgeon and one needs to avoid wrong osteotomy cuts. To this end, the inventor has developed an olecranon osteotomy clamp 400 which is also a surgical guide, see FIG. 10. The clamp is temporarily affixed to the ulna 30 at the proximal end 32 thereof which includes the olecranon, whereafter a saw is used to cut the desired portion of the olecranon utilizing the jaws of the clamp 400 as a guide.

Olecranon osteotomy clamp 400 includes a first arm 402 and a second arm 404, each having a handle 406, 408. Each arm has a jaw 410, 412 at an end opposite from the handle that forms a surgical guide for a surgical saw 460.

In one embodiment, jaws 410, 412 form surgical guide 420 at the end of each arm 402, 404. The arms transition from a relatively linear segment to inward projecting surgical guide 420. Guide 420 can be used to make a chevron type or straight osteotomy.

The olecranon osteotomy clamp 400 allows the surgeon to approach the ulna from both sides.

Locking mechanism 430 holds the surgical guides 420 in a desired position. Locking mechanism 430 preferably incudes teeth 432 or some other functionally equivalent structure on each arm of the mechanism that are releasably interlockable. The inset b) of FIG. 10 shows the olecranon after a chevron type osteotomy.

### Olecranon Plate System

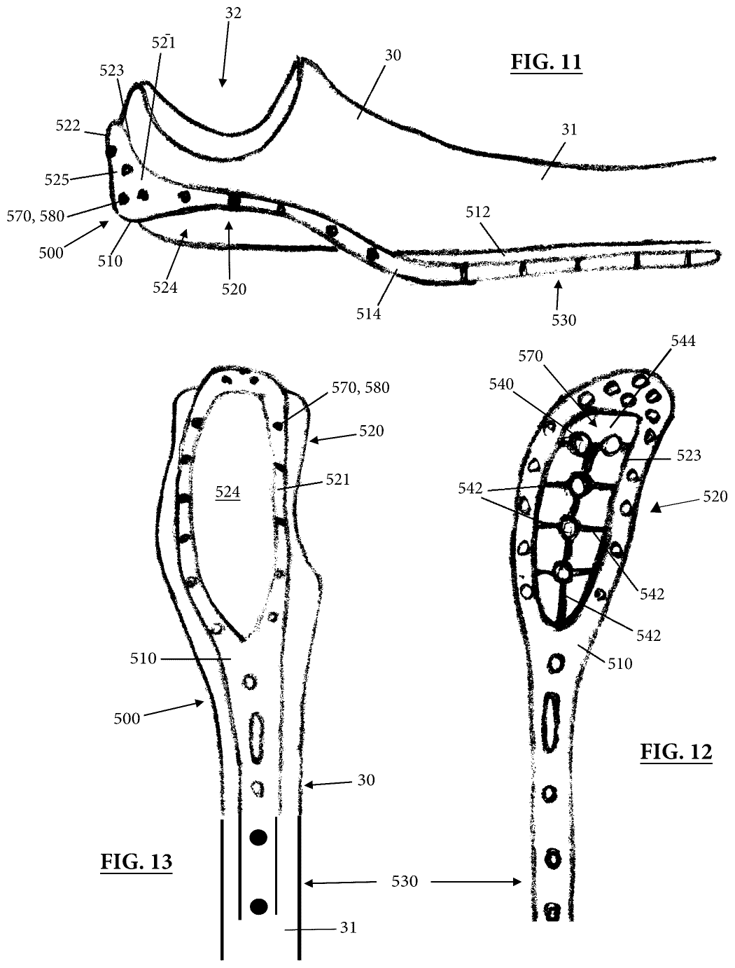

FIGS. 11, 12 and 13 illustrate an olecranon plate system 500 including an olecranon plate 510 and fasteners 580 which affix the plate 510 to the proximal end 32 of ulna 30. Olecranon plate 510 has a head segment 520 at the proximal end thereof and a stem segment 530 at a lower or second end. The stem segment 530 is connected to and extends from head segment 520. Plate 510 has an inner surface 512 adapted to engage bone of the ulna 30 and an outer surface 514 opposite the inner surface.

Head segment 520 includes an outer rim 521 having a head inner surface 523 and a head outer surface 522, with a central opening 524 located within the head inner surface 523. The outer rim 521 includes an olecranon cradle 525 adapted to be situated at the head of the olecranon, such as illustrated in FIGS. 11 and 13. The olecranon cradle 525 includes a plurality of apertures 570, each adapted for receiving a fastener 580. A plurality of apertures 570 are present in the outer rim and spaced about the central opening which provides the surgeon multiple options for securing the olecranon plate 510 to the ulna 30.

As illustrated in FIG. 12, a web 540 is present in the central opening 524 in some embodiments. Web 540 includes a plurality of relatively thin connecting filaments such as wires 542 that connect the web 540 to the inner surface of the rim. The filament can have any desired cross-sectional shape such as round, flat, square, rectangular, etc. The connecting filaments can have the same thickness as the rest of the plate in a preferred embodiment. In other embodiments, the connecting filaments can be thinner than dimensions of the plate between an inner surface an outer surface.

The web also includes a plurality of apertures 570 adapted for receiving fasteners 580. At least one portion of the web 540 can be removed from the plate by cutting the web filaments or wires 542.

Web 540 includes a central longitudinal segment 544, generally located in the middle part of central opening 524, including a plurality of apertures 570 having perimeters connected by the web filaments or wires 542. At least one of the filaments or wires 542 is connected to the outer rim 521 opposite of the location of the olecranon cradle 525, in particular to the head inner surface of the outer rim 521. In a preferred embodiment, the central longitudinal segment 544 has a plurality of filaments or wires 542 connecting the web to the inner surface of the rim laterally in relation to the central longitudinal segment 544.

The stem segment includes a plurality of apertures 570 adapted for receiving fasteners 580. In a preferred embodiment, the width of the stem segment 530 is narrower than a width of the head segment 20.

In some embodiments, at least two of the plurality of apertures 570 on the outer rim 521 have a fastener axis disposed generally between 70 and 110°, desirably between 80 and 100° and preferably about 90°, as compared to at least two of the plurality of apertures on the stem segment.

Olecranon plate 510 preferably has a thickness that ranges between 2.7 and 3.5 mm in one embodiment.

The common issues associated with olecranon fracture in the prior art are the use of relatively bulky or prominent hardware and also wound complication related to a plate that sits on the olecranon. The unique design of the olecranon plate of the present invention decreases wound complications by providing a plate having the central opening. When the web 540 or a portion of the web 540 are present, the flexibility and design allows for tailoring desired attachment to a patient.

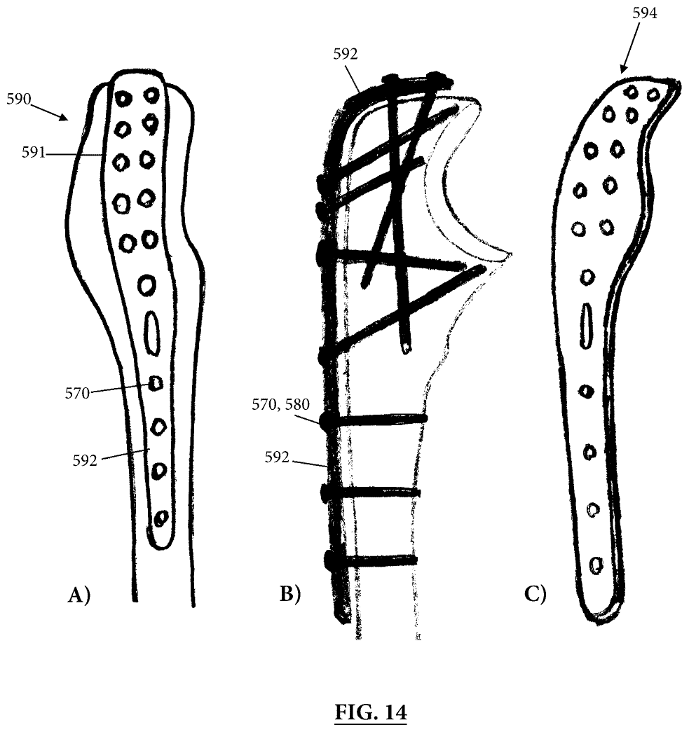

FIG. 14 shows alternative olecranon plate designs that are suitable for use in the kit of the present invention. FIGS. 14A) and B) illustrate one design of an olecranon plate 590 having a head segment 591 and a stem segment 592. A plurality of apertures 570 are present which can be used to affix the plate to the ulna with fasteners 580. FIG. 14C shows an alternative embodiment of an olecranon plate 594 that provides greater coverage area of the ulna as compared to plate 590. Plate 590 has different screw projection allowing to capture olecranon tip and coronoid fracture.

### Coronoid Clamp

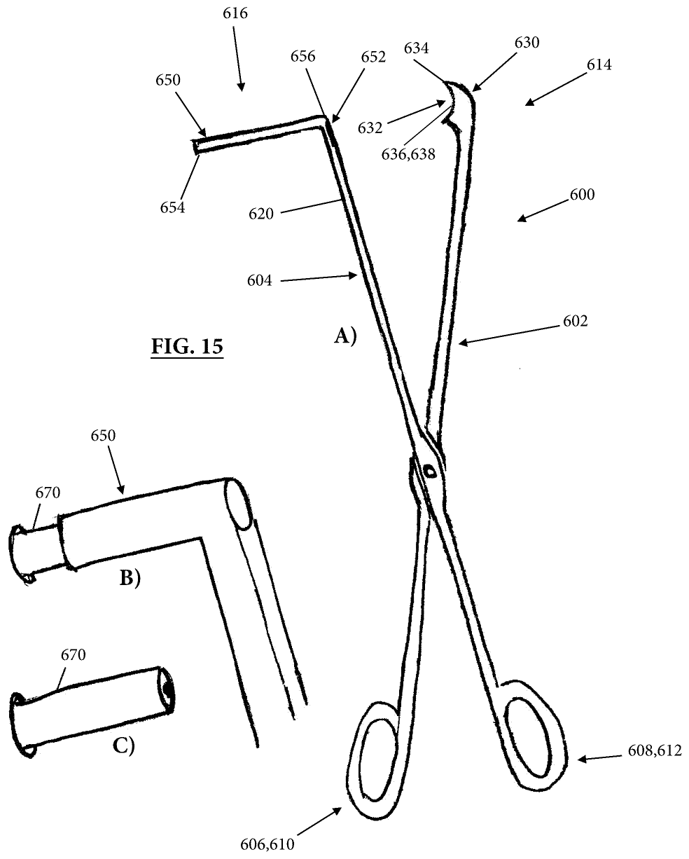

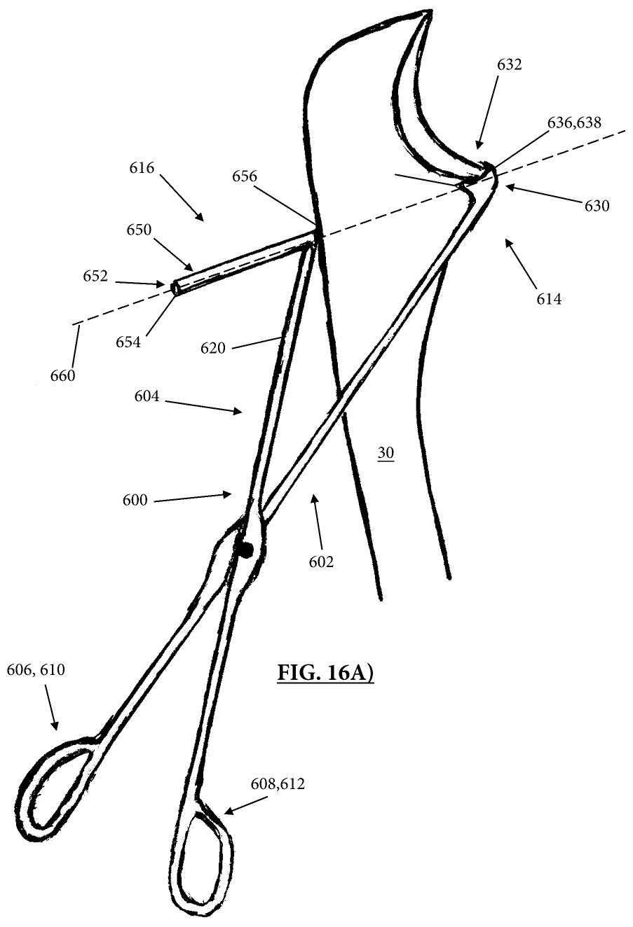

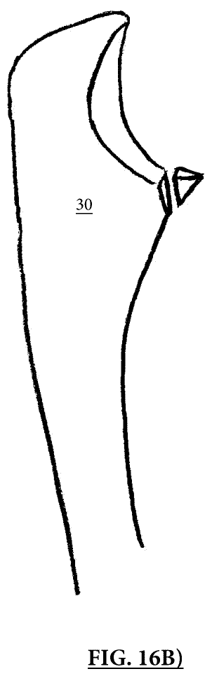

A surgical clamp 600 for the ulna 30, suitable for use in reducing a coronoid fracture, is shown in FIG. 15 A) and FIG. 16 A). A coronoid fracture is often common in an elbow fracture dislocation. Fixing a coronoid fracture such as shown in FIG. 16 B) is a challenge for orthopedic surgeons. Difficulty arises in reducing the coronoid fracture, and keeping it reduced while fixing it. The surgical clamp 600 solves this problem.

Ulna surgical clamp 600 includes a first arm 602 and a second arm 604. Each arm 602, 604 has a handle at a first end 606, 608. First arm 602 has a head 630 at a second end 614 opposite the first end 610. The head includes a coronoid cup 632 adapted to accommodate a portion of the coronoid of the ulna 30, namely the fractured portion of the coronoid as well as an unfractured portion in certain embodiments. The coronoid cup 632 has rim 634 and a base 636 recessed within the rim. In a preferred embodiment the base 636 includes at least one curved portion depending from the rim 634 adapted to accommodate a broken piece of the coronoid. In some embodiments, the head 630 is shaped similarly to a spoon.

Second arm 604 has a hollow, elongated drill guide tube 650 having a bore 652 extending therethrough at a second end 652 of the second arm 604. Drill guide tube 650 has a first end 654 and a second end 656, with a length extending therebetween. The tube 650 has a longitudinal length along a longitudinal axis 660 of the drill guide tube 650 that can be any lengths and preferably from about 1 cm to about 5 cm. In order to accommodate one or more of a K-wire and a screw, such as a cannulated screw, the bore has a diameter that can be any diameter and preferably from about 1 mm to about 5 mm.

In an important aspect of the invention, the drill guide tube 650 is connected to longitudinal segment 620 to provide a desired alignment that allows drilling through the ulna 30 and connection it to the coronoid fracture. In order to provide a desired alignment, in one preferred embodiment, an imaginary line extending through longitudinal axis 660 of the drill guide tube 650 extends to the base 636 of the head 630 of the first arm 602, with the first arm 602 located at a first position with respect to the second arm 604, such as shown in FIG. 16 A). In order to accomplish desired alignment, in one embodiment, an angle between the longitudinal segment 620 of second arm 604 and a longitudinal axis 660 of the hollow elongated drill guide tube 650 ranges generally between 60° and 120°, and preferably between 80° and 100°.

One method of surgery utilizing the surgical clamp 600 is as follows. Head 630 including the coronoid cup 632 is used to reduce the fracture of the coronoid process. The second arm 604 is then manipulated to adjust the drill guide tube 650 so that it contacts a desired area on the opposite side of the ulna 30 as the coronoid, see FIG. 16A. A K-wire 670 is advanced through drill guide tube 650 to a desired point. The length of a screw is measured. Thereafter, an inner tube is removed and drilled over the K-wire. The drill is then removed leaving the K-wire. The screw is inserted through the drill guide tube 650. Thereafter, the K-wire 670 and surgical clamp 600 are removed see FIGS. 15 B) and 15 C). In alternative embodiments, a solid screw can be used instead of a cannulated screw.

### Other Features and Additional Embodiments

In one embodiment of the invention, two or more of the devices disclosed herein are packaged together and sold as a kit. It is also important to understand that each of the devices may be sold independently as well for certain applications. Indicia may be utilized to easily identify one or more of the devices with respect to end use application or target an anatomical location. Various indicia may also be utilized to identify a particular size, a device, or orientation. Indicia may include one or more of different letters, numbers, and colors, etc.

In a preferred embodiment of the present invention, the various bone plates of the system of the present invention are provided in a shape that is substantially similar to a particular region of bone to which the plate is to be connected. In this manner, a surgeon may only need to adjust the shape of a particular plate a small amount before application. By providing plates that are already shaped, matching a bone and a plate may reduce the amount of time spent manipulating a fracture prior to applying the plate.

In order to instruct a surgeon with respect to the end uses of the plates and devices of the invention, the kits of the invention may include reference materials including instructions as to how the devices may be used, definitions of indicia, etc. The kit may also include a plurality of different size fasteners such as bone screws, as well as temporary materials, such as K-wires or the like.

i. An elbow stabilization system, comprising:
a humerus plate adapted to be connected to an external surface at a distal end of the humerus, wherein the plate has an upper proximal segment that includes a plurality of apertures adapted for fasteners that connect the humerus plate to the humerus,
an adjustable link hingedly connected to the humerus plate and having a hinge adapted to be a located adjacent where the lateral collateral ligament attaches to the humerus, wherein the adjustable link is independently adjustable in length and at least two different angles and also able to be subsequently locked in length and angle after a desired length and angle have been established; and
an ulna plate system comprising an inner plate and an outer plate each including a pair of apertures disposed at opposite ends of the plate and adapted for fasteners to fix the plates to the ulna, wherein an ulna end of the adjustable link is fixedly connectable to the ulna inner and outer plates.

ii The elbow stabilization system according to i, wherein the adjustable link comprises two separate ball and socket assemblies comprising a first ball, a first socket, a second ball and a second socket.

iii. The elbow stabilization system according to ii, wherein a slot is present between the inner plate and outer plate that accepts and fixes the ulna end of the adjustable link therebetween, wherein a first pin is connected to the first socket and a second pin is connected to the second socket and includes the ulna end of the adjustable link, wherein the first pin is hingedly connected to the humerus plate and wherein the second pin is fixable in the slot of the ulna plate system.

iv. The elbow stabilization system according to iii, wherein a connecting link is provided having the first ball at a first end and the second ball at a second end.

v. The elbow stabilization system according to iii, wherein each socket includes a bore that accepts a fastener that fixes the socket to the ball.

vi. The elbow stabilization system according to iii, wherein the hinged connection is present between the humerus plate and a first end of the first pin to let the elbow rotate from extension to flexion around an isometric axis of rotation.

vii. The elbow stabilization system according to claim 1, wherein the humerus plate consists of two apertures for fasteners that connect the humerus plate to the humerus.

viii. A distal humerus lateral plate system, comprising:
the plate having a longitudinal length and a width narrower than the length, the plate having an upper proximal first end and a lower distal second end, wherein a lower portion of the plate curves outwardly to adapt to a contour of the humerus above a lateral epicondyle of the humerus, wherein the second end of the plate includes an inwardly curved section adapted to the contour of the lateral epicondyle,
wherein the plate has a plurality of vertically spaced apertures for fasteners along the longitudinal length of the plate.

ix. The distal humerus lateral plate according to viii, wherein the second end of the plate has a posterior extension adapted to a contour of a posterior part of the humerus, wherein the posterior extension includes a plurality of spaced apertures for fasteners.

x. The distal humerus lateral plate according to ix, wherein the posterior extension includes three sets of two apertures vertically spaced along the longitudinal length thereof.

xi. A distal humerus medial plate system, comprising:
the plate having a longitudinal length and a width narrower than the length, the plate having an upper proximal first end and a lower distal second end, wherein a lower portion of the plate curves outwardly to adapt to a contour of the humerus above the medial epicondyle of the humerus, and wherein the plate has a plurality of vertically spaced apertures for fasteners along the longitudinal length of the plate.

xii. The distal humerus medial plate system according to xi, wherein the second end of the plate has a posterior extension adapted to a contour of a lower section of the medial epicondyle, and wherein the extension includes at least one aperture for a fastener.

xiii. A distal humerus plate set comprising the lateral plate system of viii and the medial plate system of xi.

xiv. A surgical drill guide for the humerus, comprising:
a first frame having a first end having a hollow elongated drill guide having a bore extending therethrough, and
a second frame having a first end having a point that aligns with the bore extending through the drill guide,
wherein the drill guide includes a locking mechanism that fixes the distance between the drill guide and the point, wherein the locking mechanism is releasable after use in order to reposition the first frame with respect to the second frame.

xv. The surgical drill guide according to xiii, wherein the first frame includes a body extending substantially perpendicular to the longitudinal length of the drill guide, wherein an arm extends outwardly from the body, with the arm having a longitudinal length, wherein the second frame includes a body extending substantially perpendicular to the longitudinal length of the drill guide, and wherein an arm extends outwardly substantially perpendicular from the body, with the arm having a longitudinal length, wherein the first frame arm and second frame arm are telescopically mated.

xvi. The surgical drill guide according to xv, wherein the locking mechanism includes teeth on each of the first frame locking mechanism arm and second frame locking mechanism arm that interlock.

xvii. The surgical drill guide according to xvi, wherein the drill guide includes two telescoping connections between arms of the first frame body and second frame body.

xviii. The surgical drill guide according to xiiii, wherein the drill guide is hingedly connected to a frame body.

xix. An olecranon osteotomy clamp and guide, comprising:
a first arm hinged to a second arm,
wherein each arm has a handle,
wherein each arm has a jaw at an end opposite from the handle, which provide a guide for both straight and chevron osteotomy; and
a locking mechanism that secures the arms in a fixed position.

xx. A olecranon plate system, comprising:
an olecranon plate having a head segment, a stem segment connected to and extending from the head segment, an inner surface adapted to engage bone of an ulna, and an outer surface opposite the inner surface,
wherein the head segment includes an outer rim having a head inner surface, a head outer surface and a central opening within the head inner surface,
wherein the outer rim includes an olecranon cradle adapted to be situated at the head of the olecranon, wherein the olecranon cradle includes a plurality of apertures each adapted for receiving a fastener, wherein a plurality of apertures are present in the outer rim spaced about the central opening,
wherein a web is present in the central opening, with the web having a plurality of wires that connect the web to the inner surface of the rim, wherein the web includes a plurality of apertures adapted for receiving the fasteners, wherein at least one portion of the web can be removed from the plate by cutting the web wires, and
wherein the stem segment includes a plurality of apertures adapted for receiving fasteners.

xxi. The olecranon plate system according to xx, wherein the web includes a central longitudinal segment including a plurality of apertures having perimeters connected by wires, and wherein at least one of the wires is connected to the outer rim opposite of the location of the olecranon cradle.

xxii. The olecranon plate system according to xxi, wherein the central longitudinal segment has a plurality of wires connecting the web to the inner surface of the rim laterally in relation to the central longitudinal segment.

xxiii. The olecranon plate system according to xx, wherein at least two of the plurality of apertures on the outer rim have a fastener axis disposed about 90° as compared to at least two of the plurality of apertures on the stem segment and wherein a width of the stem segment is narrower than a width of the head segment.

xxiv. A surgical clamp for the ulna suitable for use in reducing a coronoid fracture, comprising:
a first arm hinged to a second arm, with each arm having a handle at a first end,
wherein the first arm has a head at a second end opposite the first end, wherein the head includes a coronoid cup adapted to accommodate a portion of the coronoid, wherein the cup has a rim and a base recessed within the rim,
wherein the second arm has a hollow elongated drill guide tube having a bore extending therethrough at a second end thereof, and
wherein an imaginary line extending through a longitudinal axis of the drill guide tube also extends to the base of the head of the first arm with the first arm located at a first position with respect to the second arm.

xxv. The surgical clamp according to xxiv, wherein the hollow elongated drill guide tube has a first end and a second end, wherein the second arm has a longitudinal segment extending from a location where the first arm is hinged to the second arm, and wherein the second end of the drill guide tube is connected to the longitudinal segment.

xxvi. The surgical clamp according to xxv, wherein an angle between the longitudinal segment and the longitudinal axis of the hollow elongated drill guide ranges between 60° and 120°.

xxvii. The surgical clamp according to xxiiii, wherein the base includes at least one curved portion depending from the rim and is adapted to accommodate a broken piece of the coronoid.

xxviii. The surgical clamp guide according to xxvi, wherein the hollow elongated drill guide tube has a length with respect to the longitudinal axis that ranges from about 1 cm to about 5 cm.

xxix. The surgical clamp according to xxviii, wherein the tube bore has a diameter that ranges from about 1 mm to about 5 mm.

xxx. An elbow surgical repair kit, comprising: the components set forth in i, viii, xiv, xix, xx, and xxiv.

xxxi. The kit according to xxx, further including a second olecranon plate system, comprising:
an olecranon plate having a head segment, a stem segment connected to and extending from the head segment, an inner surface adapted to engage bone of an ulna, and an outer surface opposite the inner surface,
wherein the olecranon plate has a longitudinal length,
wherein with respect to the longitudinal length, the head segment is wider than the stem segment,
wherein the stem segment includes longitudinal spaced apertures, wherein the head segment includes a plurality of longitudinally spaced apertures, and
wherein the head segment includes at least one pair of apertures laterally disposed with respect to the longitudinal length.

For the avoidance of doubt, the apparatus and devices of the present invention encompass all possible combinations of the components, including various ranges of said components, disclosed herein. It is further noted that the term ‘comprising’ does not exclude the presence of other elements. However, it is also to be understood that a description of an apparatus comprising certain components also discloses a product consisting of these components. Similarly, it is also to be understood that a description of a process comprising certain steps also discloses a process consisting of these steps.

In accordance with the patent statutes, the best mode and preferred embodiment have been set forth; the scope of the invention is not limited thereto, but rather by the scope of the attached claims.

What is claimed is:

1. An elbow stabilization system, comprising:
a humerus plate adapted to be connected to an external surface at a distal end of the humerus, wherein the plate has an upper proximal segment that includes a plurality of apertures adapted for fasteners that connect the humerus plate to the humerus,
an adjustable link hingedly connected to the humerus plate and having a hinge adapted to be a located adjacent where the lateral collateral ligament attaches to the humerus, wherein the adjustable link is independently adjustable in length and at least two different angles and also able to be subsequently locked in length and angle after a desired length and angle have been established; and
an ulna plate system comprising an inner plate and an outer plate each including a pair of apertures disposed at opposite ends of the plate and adapted for fasteners to fix the plates to the ulna, wherein an ulna end of the adjustable link is fixedly connectable to the ulna inner and outer plates.

2. The elbow stabilization system according to claim 1, wherein the adjustable link comprises two separate ball and socket assemblies comprising a first ball, a first socket, a second ball and a second socket.

3. The elbow stabilization system according to claim 2, wherein a slot is present between the inner plate and outer plate that accepts and fixes the ulna end of the adjustable link therebetween, wherein a first pin is connected to the first socket and a second pin is connected to the second socket and includes the ulna end of the adjustable link, wherein the first pin is hingedly connected to the humerus plate and wherein the second pin is fixable in the slot of the ulna plate system.

4. The elbow stabilization system according to claim 3, wherein a connecting link is provided having the first ball at a first end and the second ball at a second end.

5. The elbow stabilization system according to claim 3, wherein each socket includes a bore that accepts a fastener that fixes the socket to the ball.

6. The elbow stabilization system according to claim 3, wherein the hinged connection is present between the humerus plate and a first end of the first pin to let the elbow rotate from extension to flexion around an isometric axis of rotation.

7. The elbow stabilization system according to claim 1, wherein the humerus plate consists of two apertures for fasteners that connect the humerus plate to the humerus.

8. A distal humerus lateral plate system, comprising:
the plate having a longitudinal length and a width narrower than the length, the plate having an upper proximal first end and a lower distal second end, wherein a lower portion of the plate curves outwardly to adapt to a contour of the humerus above a lateral epicondyle of the humerus, wherein the second end of the plate includes an inwardly curved section adapted to the contour of the lateral epicondyle,
wherein the plate has a plurality of vertically spaced apertures for fasteners along the longitudinal length of the plate.

9. The distal humerus lateral plate according to claim 8, wherein the second end of the plate has a posterior extension adapted to a contour of a posterior part of the humerus, wherein the posterior extension includes a plurality of spaced apertures for fasteners.

10. The distal humerus lateral plate according to claim 9, wherein the posterior extension includes three sets of two apertures vertically spaced along the longitudinal length thereof.

11. A distal humerus medial plate system, comprising:
the plate having a longitudinal length and a width narrower than the length, the plate having an upper proximal first end and a lower distal second end, wherein a lower portion of the plate curves outwardly to adapt to a contour of the humerus above the medial epicondyle of the humerus, and wherein the plate has a plurality of vertically spaced apertures for fasteners along the longitudinal length of the plate.

12. The distal humerus medial plate system according to claim 11, wherein the second end of the plate has a posterior extension adapted to a contour of a lower section of the medial epicondyle, and wherein the extension includes at least one aperture for a fastener.

13. A distal humerus plate set comprising the lateral plate system of claim 8 and
a distal humerus medial plate system, comprising:
the plate having a longitudinal length and a width narrower than the length, the plate having an upper proximal first end and a lower distal second end, wherein a lower portion of the plate curves outwardly to adapt to a contour of the humerus above the medial epicondyle of the humerus, and wherein the plate has a plurality of vertically spaced apertures for fasteners along the longitudinal length of the plate.
